(19) United States
(12) Patent Application Publication
(10) Pub. No.: US 2012/0238736 A1
(43) Pub. Date: Sep. 20, 2012

(54) DEVICE FOR SHEARING NUCLEIC ACIDS AND PARTICULATES

(76) Inventor: THOMAS W. HARDING, Wilmington, DE (US)

(21) Appl. No.: 13/420,171

(22) Filed: Mar. 14, 2012

Related U.S. Application Data
(60) Provisional application No. 61/452,683, filed on Mar. 15, 2011.

(51) Int. Cl. C07H 21/00; B06B 1/00

(52) U.S. Cl. 536/23.1; 422/128

(57) ABSTRACT

Disclosed herein is a device for shearing nucleic acids and particulates in a syringe or vial comprising an ultrasonic transducer attached to a solid cylindrical horn comprising a tip shaped to engage the profile of the tip of a syringe or vial. The horn tuned for maximum amplitude vibration at its tip.

### CROSS-REFERENCE TO RELATED APPLICATION

This application claims benefit of U.S. Provisional Patent Application No. 61/452,683, filed Mar. 15, 2011, which is incorporated by reference herein in its entirety.

### FIELD OF INVENTION

The field relates to devices for shearing nucleic acids and particulates in syringe or vial.

### BACKGROUND OF INVENTION

The analysis of samples for microorganisms, such as bacteria, is important for public health. Foods grown, purchased, and consumed by the general population may contain or acquire microorganisms, which flourish or grow as a function of the environment in which they are located. This growth may lead to accelerated spoilage of the food product or the proliferation of pathogenic organisms, which may produce toxins or allergens.

In the course of analyzing samples from microorganisms in foods, one must contend with large nucleic acid molecules and large particulates that can hinder later detection steps. As such, there is a need for a device that can reduce the size of these large nucleic acid molecules and particulates while retaining the detectability of the nucleic acids.

### SUMMARY OF INVENTION

One aspect is for a device for shearing nucleic acids and particulates in a syringe or vial comprising an ultrasonic transducer attached to a solid cylindrical horn, said horn comprising a tip shaped to engage the profile of the tip of a syringe or vial and said horn tuned for maximum amplitude vibration at the tip of the horn.

Another aspect is for a device for shearing nucleic acids and particulates in a syringe or vial comprising an ultrasonic transducer; a solid cylindrical horn, said horn comprising a tip shaped to engage the side profile of a syringe or vial and said horn tuned for maximum amplitude vibration at the tip of the horn; a tuned booster detachably engaged to the ultrasonic transducer and the solid cylindrical horn; and an anvil mated to the horn, said anvil being capable of clamping the horn to the syringe or vial.

Other objects and advantages will become apparent to those skilled in the art upon reference to the detailed description that hereinafter follows.

### BRIEF DESCRIPTION OF THE FIGURES

FIG. 1 shows a first embodiment of a device for shearing nucleic acids and particulates.

FIG. 2 shows a second embodiment of a device for shearing nucleic acids and particulates.

### DETAILED DESCRIPTION OF PREFERRED EMBODIMENTS

Applicant specifically incorporates the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

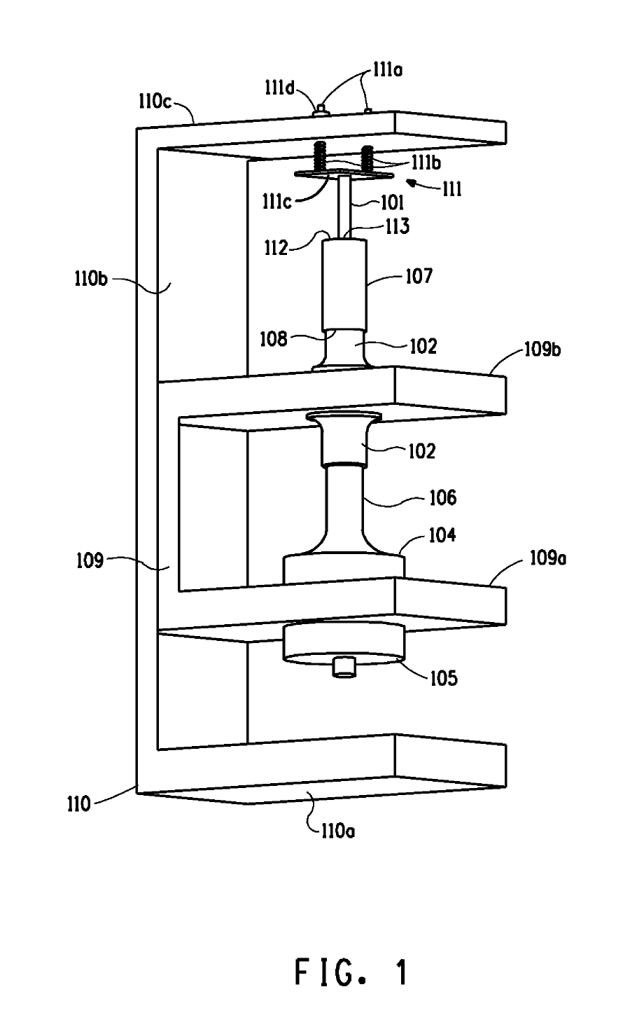

FIG. 1 shows a first embodiment of the device for shearing nucleic acids and particulates. Referring to FIG. 1, the ultrasonic transducer 104, 105, 106 comprises a Piezoelectric crystal stack (not shown) inside a housing 104 capable of oscillating in response to energy input from a power supply, not shown. The vibratory energy from the piezoelectric crystal stack is transmitted to the element 106 and is substantially distributed in a direction towards the syringe or vial 101. The ultrasonic transducer 104, 105, 106 can be piezoelectric transducer or a magnetostrictive transducer. A piezoelectric transducer can be manufactured from any common piezoelectric material, such as lead zirconate titanate which expands and contracts when provided with the appropriate AC electrical voltage at the correct frequency. A magnetostrictive transducer is essentially an electromagnet made of a heavy nickel or alloy core which is wound with wire. As electrical current is pulsed through the wires, the core vibrates at a frequency which matches the output frequency of the ultrasonic generator. Preferably, the ultrasonic transducer 104, 105, 106 is a piezoelectric transducer. More preferably, the ultrasonic transducer is a Dukane Corp. (St. Charles, Ill.) piezoelectric device, model 41C28, that is fed an AC voltage of a particular frequency (for example, 40 kHz) by a power supply (Dukane Corp. Model 40A351). The electrical energy is converted into mechanical vibration that travels from the transducer to the horn and sets up a standing wave in the stack of transducer, booster (when present), and horn with the stack vibrating in the axial direction.

The optional tuned booster 102 amplifies or modifies the vibrations generated by the ultrasonic transducer 104 to the solid cylindrical horn 107. When present, the tuned booster 102 is detachably mounted to the front end 106 of the ultrasonic transducer 104, and detachably mounted to the back end portion 108 of the solid cylindrical horn 107. The ultrasonic energy transmitted through the tuned booster 102 to the solid cylindrical horn 107 propagates in the particulate sample containing nucleic acids in the syringe or vial. As a result, large nucleic acids and/or particulates in the particulate sample are sheared by the ultrasonic energy. The tuned booster section is typically made of titanium or aluminum.

The end of the solid cylindrical horn 107 is an antinode in the vibration and vibrates with maximum amplitude at the tip 112. In a preferred embodiment, the tip 112 contains a concave syringe or vial receiving section 113 designed to hold the syringe or vial 101 in a vertical orientation. In a preferred embodiment, the syringe or vial receiving section 113 can be machined to match the shape of the tip of the syringe or vial 101 to ensure proper seating of the syringe or vial 101 in the syringe or vial receiving section 123 and to ensure good energy transfer from the horn tip 112 to the vial or syringe 101.

The solid cylindrical horn 107 comprises a stiff or rigid material and may be formed of a variety of materials including, without limitation, aluminum, titanium, and MONEL® (nickel/copper alloy). Preferably, the solid cylindrical horn 107 is aluminum.

The device is supported by a base plate 110. Preferably, the base plate 110 comprises a base 110a in an orthogonal relationship to a side 110b which in turn is in an orthogonal relationship to a top 110c resulting in the base 110a and the top 110c being essentially parallel to each other. When using the device, the base 110a acts as the bottom of the device and can be attached to a further support mechanism (for example, a table) to ensure stability of the device when in operation. The base material can be aluminum or any other material that provides structural rigidity.

Attached to the side 110b of the base 110, preferably removably attached, is a transducer mounting yoke 109. As shown in FIG. 1, the ultrasonic transducer 104 can be mounted in the transducer mounting yoke 109. Regardless of arrangement, the ultrasonic transducer 104 can be either removably or fixedly attached to the transducer mounting yoke 109. In embodiments lacking a tuned booster 102, the active element 106 of the ultrasonic transducer 104 passes through an opening constructed in the top 109b of the transducer mounting yoke 109 to the active element's 106 attachment to the solid cylindrical horn 107. In embodiments utilizing a tuned booster 102, for example as shown in FIG. 1, it is the tuned booster 102 that passes through an opening constructed in the top 109b of the transducer mounting yoke 109 to the tuned booster's 102 attachment to the solid cylindrical horn 107. The transducer mounting yoke components are typically made of aluminum or steel but any sufficiently rigid material can be used.

If a vial is used, the horn tip 112 can be machined to match the profile of the end of the vial. For instance, if a round bottom vial is used, then a hemispherical depression can be machined in the horn to match the vial profile to ensure maximum contact and hence maximal energy transfer to the vial contents. To improve energy transfer, fluids such as oils can be used to improve sound coupling. The tip 112 could also be coated with a thin layer of rubber or other conformable material to improve the coupling of sound energy into the syringe or vial 101.

In a preferred embodiment of the first device, a one inch diameter cylindrical horn was used with a tip that was machined to match the profile of the end of a plastic syringe or plastic vial. This is done to ensure maximum contact between the horn and the syringe to maximize the transfer of sound energy into the fluid. For instance, the plastic 5 cc syringe that was used had a shallow conical end and a standard tapered Luer tip. A shallow conical depression was machined into the horn end to match the syringe and a 3/16 inch diameter hole was drilled axially to accommodate the Luer tip and a metal plug. In some embodiments, hand pressure was used to push the liquid filled syringe onto the horn end. Preferably though, and as shown in FIG. 1, a spring loaded device 111 was used to push the syringe or vial 101 onto the horn tip 112. The spring loaded device 111 is employed to ensure repeatable pressure and orientation of the syringe or vial 101.

The spring loaded device 111 is removably attached to the top 110c of the base plate 110, preferably through a bolting mechanism 111a. In FIG. 1, the spring loaded device 111 is shown as having two springs 111b, though one spring or three springs or more are acceptable. The bolting mechanism 111a passes through the top 110c of the base plate 110 and is attached, preferably removably attached, to a pressure plate 111c. The pressure plate 111c engages the syringe or vial 101 to push the syringe or vial 101 onto the horn tip 112. The spring 111b is fitted on the bolting mechanism 111a prior to the bolting mechanism's 111a insertion through the underside of the top 110c of the base plate 110. A bolt 111d or similar locking device is used to secure the bolting mechanism 111a to the top 110c of the base plate 110 and to ensure proper distance between the pressure plate 111c and the tip 112 of the solid cylindrical horn 107.

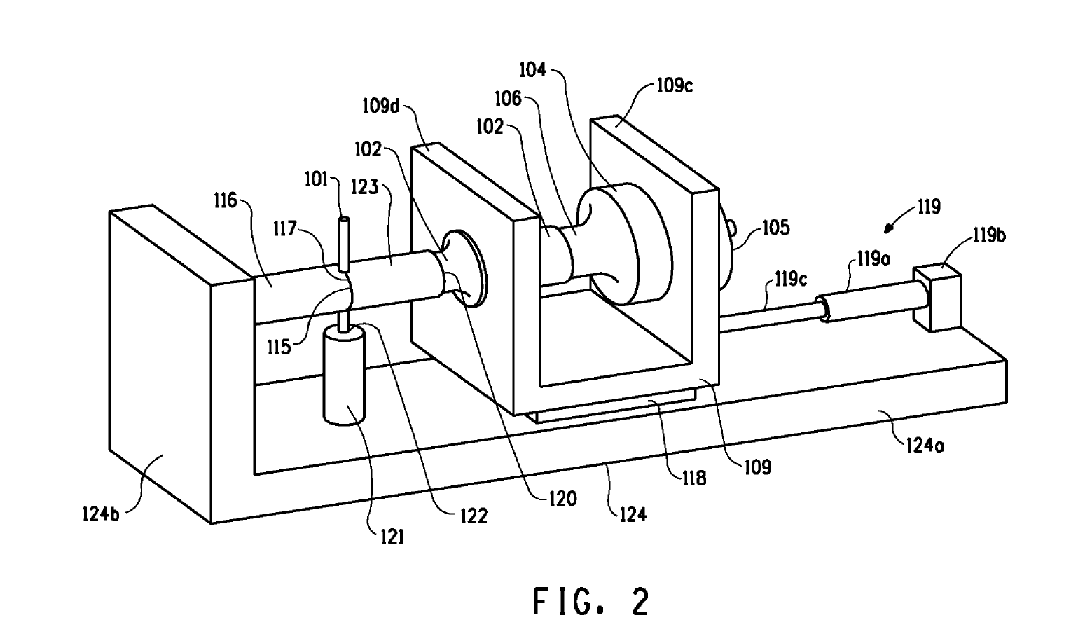

FIG. 2 shows a second embodiment of the device for shearing nucleic acids and particulates. The ultrasonic transducer 104, 105, 106 of the second embodiment is as described above for the first embodiment.

The solid cylindrical horn 123 of the second embodiment is shaped such that its end opposite that in contact with the ultrasonic booster 102 is capable of engaging the profile of the syringe or vial 101. This tip 115 of the solid cylindrical horn 123 is also shaped so that it does not contact the front end portion 117 of the anvil 116. As with the first embodiment, the solid cylindrical horn 123 of the second embodiment comprises a stiff or rigid material with low acoustic loss and may be formed of a variety of materials including, without limitation, aluminum, titanium, or MONEL® (nickel/copper alloy). Preferably, the solid cylindrical horn 123 is aluminum.

The tuned booster 102 is detachably mounted to the transducer front end 106 of the ultrasonic transducer 104, 105, 106, and detachably engages the back end portion 120 of the solid cylindrical horn 123. The ultrasonic energy transmitted through the tuned booster 102 to the solid cylindrical horn 123 propagates in the particulate sample containing nucleic acids in the syringe or vial. As a result, large nucleic acids and/or particulates in the particulate sample are sheared by the ultrasonic energy.

The second embodiment device is supported by a base plate 124. Preferably, the base plate 124 comprises a base 124a in an orthogonal relationship to a side 124b. When using the device, the base 124a acts as the bottom of the device and can be attached to a further support mechanism (for example, a table) to ensure stability of the device when in operation. The anvil 116 is attached, preferably removably attached, to side 124b of the base plate 124 at the opposite end of the anvil 116 to the front end portion 117. The anvil can be made of, e.g., aluminum or titanium.

As shown in FIG. 2, the ultrasonic transducer 104 can be mounted in the transducer mounting yoke 109. The ultrasonic transducer 104 can be either removably or fixedly attached to the transducer mounting yoke 109. The tuned booster 102 passes through an opening constructed in the horn side 109d of the transducer mounting yoke 109 to the tuned booster's 102 attachment to the solid cylindrical horn 123.

In some embodiments (not shown in FIG. 2), the transducer mounting yoke 109 has its transducer side 109c and its horn side 109d split transverse to the transducer 104, and booster axis and clamp bolts are used to hold the transducer 104 between the transducer side 109c and its horn side 109d of the transducer mounting yoke 109.

A support post 121 is attached to base 124a of the base plate 124. The support post 121 contains at the end opposite of the support post's 121 attachment to the base plate 124 a concave syringe or vial receiving section 122 designed to hold the syringe or vial 101 in a vertical orientation. In a preferred embodiment, the syringe or vial receiving section 122 can be machined to match the shape of the tip of the syringe or vial 101 to ensure proper seating of the syringe or vial 101 in the syringe or vial receiving section 122.

In the second embodiment, the transducer mounting yoke 109 is not attached to the base 124. Instead, the transducer mounting yoke 109 is movable along the axis defined by the solid cylindrical horn 123 and the anvil 116. Preferably, such motion is accomplished by having the transducer mounting yoke 109 mounted on a ball slide 118 that is movably engaged with a pneumatic or hydraulic cylinder or a spring capable of supplying a clamping force between the horn and the anvil. As shown in FIG. 2, a pneumatic cylinder 119 is attached to the base 124a of the base plate 124 by a pneumatic cylinder support 119b. A cylinder body 119a extends in the direction of the bail slide 118 and is connected thereto by a plunger 119c. The ball slide 118 operates by applying air pressure to the pneumatic cylinder 119 thereby extending the plunger 119c which pushes on the ball slide and transducer mounting yoke 109.

The syringe or vial 101 containing a particulate sample containing nucleic acids is inserted into the syringe or vial receiving section 122 when the pneumatic cylinder 119 and plunger 119c are retracted. Air pressure is applied to the pneumatic cylinder 119 and the ball slide 118, transducer yoke 109 and transducer 104 are pushed forward so that the tip 115 of the solid cylindrical horn 123 engages the syringe or vial 101. Preferably, the front end portion 117 of the anvil 116 also engages the syringe or vial 101 to increase the effectiveness of ultrasonic energy transmission into the syringe or vial 101 and/or to aid in supporting the syringe or vial 101 in a vertical orientation.

In a preferred embodiment, the second device uses an ultrasonic transducer, booster and horn, and an anvil to clamp a liquid filled syringe or vial in a way that transmits sound energy into the side of the syringe or vial. The horn tip and mating anvil have a semi-cylindrical cut in their ends that is transverse to the horn and transducer axis and whose radius is matched to the syringe or vial cylindrical radius. If the syringe or vial is tapered slightly, then the cut can be tapered to match. This ensures maximum contact area with the syringe or vial and ensures maximum transfer of sound energy to the contents of the vial. The diameter of the horn is chosen to match the height of the liquid in the syringe or vial. The transducer and horn assembly is mounted to a ball slide that allows one dimensional motion. The components are further mounted on a base. A pneumatic cylinder is used to apply pressure to the transducer and horn that squeezes the syringe or vial into the fixed anvil. The horn and anvil are machined in such a way that when the vial is clamped between the horn and anvil, there is no contact between the horn and anvil. This arrangement has an advantage in that the area of contact with the syringe or vial is larger and allows for more efficient transfer of sound energy into the liquid contents compared to the first embodiment. However, if the quantity of fluid in the syringe or vial is small, less than 0.5 cc, for instance, then the first device is the better arrangement to use for sonication.

In a preferred embodiment, the transducer (e.g., Dukane Corp. model 41C28) is powered by a power supply (e.g., Dukane Model 40A351) that supplies electrical power at high voltage and at, e.g., 40 kHz frequency to the transducer via a coaxial cable. The high frequency, high voltage signal is applied to the faces of the piezoeletric crystals inside the transducer and the electrical energy is converted to mechanical vibration by the crystal stack. This vibrational energy is transferred to the end of the transducer and into the booster and horn.

The intensity of the sonic energy may vary widely. Best results will generally be achieved with an intensity ranging from about 1 W/cm² to about 30 W/cm².

The residence time of exposure of the nucleic acids and/or particulates in the syringe or vial to ultrasound is not critical, and the optimal residence time will vary according to the sample being treated. Best results, however, will generally be obtained with residence times ranging from about 1 second to about 2 minutes, preferably about 1 second to about 30 seconds.

What is claimed is:

1. A device for shearing nucleic acids and particulates in a syringe or vial comprising an ultrasonic transducer attached to a solid cylindrical horn, said horn comprising a tip shaped to engage the profile of the tip of a syringe or vial and said horn tuned for maximum amplitude vibration at the tip of the horn.

2. The device of claim 1, wherein the horn comprises a material selected from the group consisting of aluminum, titanium, and MONEL (nickel/copper alloy).

3. The device of claim 1, further comprising a tuned booster detachably engaged to the ultrasonic transducer and the solid cylindrical horn.

4. The device of claim 1, wherein the horn comprises on its surface a conformable material capable of improving coupling of sound energy into the syringe or vial.

5. The device of claim 4, wherein the conformable material is a thin layer of rubber.

6. The device of claim 1, wherein the ultrasonic transducer is piezoelectric.

7. The device of claim 1, wherein the ultrasonic transducer is magnetostrictive.

8. A device for shearing nucleic acids and particulates in a syringe or vial comprising
an ultrasonic transducer;
a solid cylindrical horn, said horn comprising a tip shaped to engage the side profile of a syringe or vial and said horn tuned for maximum amplitude vibration at the tip of the horn;
a tuned booster detachably engaged to the ultrasonic transducer and the solid cylindrical horn; and
an anvil mated to the horn, said anvil being capable of clamping the horn to the syringe or vial.

9. The device of claim 8, wherein said tip and anvil each have a semi-cylindrical cut in their respective ends that is transverse to the horn and transducer axis.

10. The device of claim 9, wherein said ultrasonic transducer is mounted on a ball slide.

11. The device of claim 10, wherein the ball slide is connected to a pneumatic or hydraulic cylinder or spring capable of supplying a clamping force between the horn and the anvil.

12. A method of sonicating the contents of a syringe or vial comprising exposing the syringe or vial to ultrasonic energy generated by the device of claim 1 or 8.
